(19) United States
(12) Patent Application Publication
(10) Pub. No.: US 2004/0260402 A1
(43) Pub. Date: Dec. 23, 2004

(54) Method of manufacturing a socket portion of a prosthetic limb

(76) Inventors: Steven E. Baldini, St. Charles, MO (US); Sidney W. Sprouse, Wildwood, MO (US); William D. Macy, St. Peters, MO (US)

Correspondence Address: Clyde L. Smith, Thompson Coburn LLP, One US Bank Plaza, St. Louis, MO, 63101, US

(21) Appl. No.: 10/600,546

(22) Filed: Jun. 20, 2003

(51) Int. Cl. A61F 2/80

(52) U.S. Cl. 623/33; 623/36; 623/901; 700/119

(57) ABSTRACT

A method of forming prosthetic limbs is disclosed. A preferred embodiment of practicing the invention comprises the basic steps of scanning a portion of a residual limb on which a prosthetic device is to be attached, creating a digital representation of a socket portion of the prosthetic device using the scanned information, and creating the socket out of physical material using a digitally controlled layered manufacturing technique driven by the digital representation of the socket.

### BACKGROUND OF THE INVENTION

Prosthetic limbs must often be customized to fit a particular wearer. More particularly, the socket portion of a prosthetic limb is often customized to conform generally to the unique shape of the wearer's residual limb to which the prosthetic limb is attached. This is done to maintain a firm connection between the prosthetic limb and the wearer's residual limb, as well as to distribute the loads transferred therebetween evenly and in a manner that is comfortable to the wearer.

Typically, the socket of a prosthetic limb is configured as a cup-shaped structure defined by a relatively thin socket wall and by a cavity that extends into the socket and is adapted for insertion of a portion of the wearer's residual limb. The interior surface of such sockets defines the cavity and is generally shaped to conform the three-dimensional contour of a portion of the residual limb to which the socket is attached. However, the contour of the inner surface of a socket is most often rectified, or altered from being fully conformal, by adding protuberances that protrude slightly from the socket wall into the socket cavity. The protuberances are designed and utilized to facilitate the transfer of loads between the socket and the residual limb and their design and placement require highly specialized expertise. The terminal edge of the cavity of a socket defines a perimeter surface and is usually contoured in a non-planer manner so that the socket provides maximum load bearing support without posing an impediment to the movement and use of the prosthetic limb by its wearer.

In addition to the socket, a prosthetic limb assembly typically also comprises a liner or sock, a pylon or appendage portion, and a pylon fitting. Liners are typically made of a to resilient material such as rubber or the like and are used as an interface between the socket of the prosthetic limb and the residual limb of the wearer. Typically, liners are cup-shaped and are configured to resiliently stretch so as to conform to a wearer's residual limb. In addition to providing a cushion between the socket of the prosthetic limb and the wearer's residual limb, an attachment member fixed to the base of the liner facilitates the attachment of the prosthetic limb to the wearer's residual limb. With the liner stretched around the residual limb, the liner cannot be easily removed without resiliently deflecting the liner material away from the residual limb. Thus, tension can be applied to the attachment connector of the liner and thereby transferred to the residual limb. Using this capability, the attachment connector is passed through an opening that extends through the wall of the socket and a ratcheting connection member, threaded nut, or other type of connection member is used to bias the residual limb into the socket by sandwiching the socket between the liner and the connection member.

Appendage portions of prosthetic limbs vary in form and purpose. If the prosthetic limb is used to replace a human leg, the appendage portion may comprise a shaft or tube with an artifical foot attached thereto that is configured and adapted to bear the load of a wearer's weight. In other situations, the pylon portion of the prosthetic limb may be purely cosmetic. Nonetheless, the pylon portion is generally releasably attached to the socket of the prosthetic limb via a fitting that is rigidly attached to the socket.

Because the present invention pertains particularly to methods of forming the socket portions of prosthetic limbs, discussion of the traditional methods of forming sockets is warranted. Traditionally, the socket of prosthetic limb is formed by first making a positive mold of at least a portion of the particular residual limb for which the socket is being made. The most common way this is done is by first making a negative mold of the residual limb using plaster or other suitable materials. A positive mold of the residual limb is then made is then made via the negative mold. Alternatively, in some cases, the residual limb is scanned in a manner to create a digital representation of the three-dimensional surface contour of the residual limb which is used to drive a digitally controlled milling machine which then cuts a positive mold of the residual limb out of any suitable material.

The next step of creating a socket typically comprises altering the surface contour of the positive mold by either adding material or by cutting or otherwise removing material from the positive mold to form the negative of the protuberances discussed above. Using this altered or rectified positive mold, a check socket is formed therearound by layering fiberglass or other suitable materials over the positive mold. The purpose of the check socket is to form an inexpensive socket for testing the fit between the socket cavity and the wearer, and thus, the check socket is typically not fully functional as a socket. If the test socket demonstrates an acceptable fit, a second positive mold of the socket cavity is formed from the test socket. During this procedure, the test socket must be destroyed. Finally, the actual socket is produced from this second positive mold, typically by layering fiberglass composite on the mold. When the fiberglass or other material has cured or hardened, the second positive mold is destroyed and removed from what has become the cavity of the finished socket.

To complete the manufacture of the socket, the perimeter edge of the socket surrounding the socket cavity is cut or trimmed to provide the socket with a suitable perimeter contour. Additionally, a hole is typically cut or formed into the base of the socket to allow the attachment connector of a liner to pass through the socket wall. In some cases, a metal or other non-fiberglass material fitting must be integrated into the fiberglass for this purpose during the step of forming the fiberglass on the mold. Finally, a fitting for securing a pylon or appendage portion of the prosthetic limb to the socket is rigidly fixed to the base of the socket.

While prior art methods of forming the sockets of prosthetic limbs have proven to be successful, the inventors of the present invention have appreciated disadvantages associated such methods. One such disadvantage is that, if the formed socket is not acceptable, many if not all of the steps of forming the socket must be repeated. This is because the positive mold used to create the socket is destroyed during the process of removing the mold from the socket. Another disadvantage is that the numerous steps and expertise require to form the sockets makes the formation of an acceptable socket costly, laborious, and time consuming. In view of these disadvantages and others appreciated by the inventors, the present invention was developed.

### SUMMARY OF THE INVENTION

The present invention overcomes many of the disadvantages associated with the formation of prosthetic limb sockets by drastically changing the way a socket is formed. The present invention also allows additional features, not previously obtainable using convention methods, to be formed in the socket of a prosthetic limb.

In a first aspect of the invention, a method of forming a prosthetic limb for attachment to a residual limb of a living animal comprises the steps of generating a digital representation of a three-dimensional surface contour, generating a digital representation of a socket of the prosthetic limb, and forming the socket out of physical material. The digital representation of the three-dimensional surface contour is dependent on a physical three-dimensional surface contour of at least a portion of the residual limb. The socket of the prosthetic limb has cavity defined by an interior surface that is defined at least partially by the digital representation of the three-dimensional contour. The formation of the socket out of physical material occurs using a digitally controlled layered manufacturing technique driven by the digital representation of the socket.

In a second aspect of the invention, a method of forming a socket of prosthetic limb and attaching the socket to a residual limb of a living animal comprises the steps of positioning a liner on at least a portion of the residual limb, marking the liner in manner indicating a preferred contour and location of a non-planer terminal edge of the socket, electronically scanning at least a portion of the liner, generating a digital representation of the socket, using a digitally controlled layered manufacturing technique to form the socket out of physical material, and attaching the socket of the prosthetic limb to the residual limb. The marking of the liner occurs when the liner is positioned on the residual limb. The electronic scanning of the portion of the liner occurs with the liner positioned on the residual limb and is performed to generate a digital representation of a three-dimensional surface contour that is dependent on a physical three-dimensional surface contour of the liner when the liner is positioned on the residual limb. The scanning also occurs in a manner such that the contour and location of the non-planer terminal edge of the socket that has been marked on the liner is identifiable in the digital representation of the three-dimensional surface contour. The digitally represented socket has a cavity defined by an interior surface and has an exterior surface and a non-planar perimeter surface. The interior surface is defined at least partially by the digital representation of the three-dimensional contour. The perimeter surface terminates the cavity and bridges the exterior and interior surfaces. The perimeter surface is also dependent upon the contour and location of the non-planer terminal edge of the socket that is identifiable in the digital representation of the three-dimensional surface contour. The digitally controlled layered manufacturing technique is driven by the digital representation of the socket to form the interior, exterior, and perimeter surfaces of the socket out of physical material. Finally, the socket of the prosthetic limb is attached to the residual limb by positioning the residual limb with the liner positioned thereon at least partially into the cavity of the socket.

In yet a third embodiment of the invention, a method of forming a socket of a prosthetic limb and attaching the socket to a residual limb of a living animal comprises the steps of positioning a liner on at least a portion of the residual limb, forming a socket, and attaching the socket of the prosthetic limb to the residual limb. The liner has an exterior surface contour when the liner is positioned on the portion of the residual limb. The socket has an exterior surface and a cavity that is defined by an interior surface. The interior surface of the socket has a contour that is dependent upon the exterior surface contour of the liner and is formed without a process of rectifying the contour for the purpose of altering the bearing characteristics between the socket and the liner. Finally, the attaching the socket of the prosthetic limb to the residual limb is performed by positioning the residual limb with the liner positioned thereon at least partially into the cavity of the socket.

While the principal advantages and features of the invention have been described above, additional features and advantages may be obtained by referring to the drawings and the detailed description of the preferred embodiment, which follow.

### BRIEF DESCRIPTION OF THE DRAWINGS

FIG. 1 is flow chart of a preferred method of practicing the invention to form the socket of a prosthetic limb.

FIG. 2 is a side elevation view of a residual limb and is shown with a liner and a band of artifacts positioned thereon.

FIG. 3 is a perspective view of a socket of a prosthetic limb formed using the preferred method of practicing the invention.

FIG. 4 is a cross-sectional view of the socket shown in FIG. 3, taken about line 4-4 of FIG. 3.

Reference characters in the written specification indicate corresponding items shown throughout the drawing figures.

### DETAILED DESCRIPTION OF THE PREFERRED EMBODIMENT OF THE INVENTION

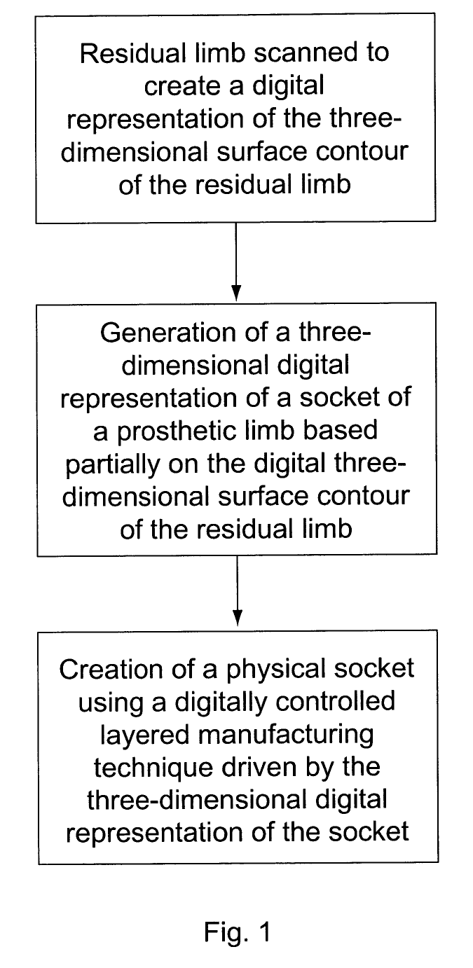

A flow chart of the basic steps of the preferred method of practicing the invention is shown in FIG. 1. As shown, the preferred method of practicing the invention comprises the basic steps of scanning a portion of a residual limb on which a prosthetic device is to be attached, creating a digital representation of a socket portion of the prosthetic device using the scanned information, and creating the socket out of physical material using a digitally controlled layered manufacturing technique driven by the digital representation of the socket. The residual limb on which the prosthetic device is to be attached is preferably a human limb. However, it should be appreciated that the preferred method of practicing the invention could also be utilized to form prosthetic limbs for many other type of animals, including but not limited to, dogs, cats, and horses.

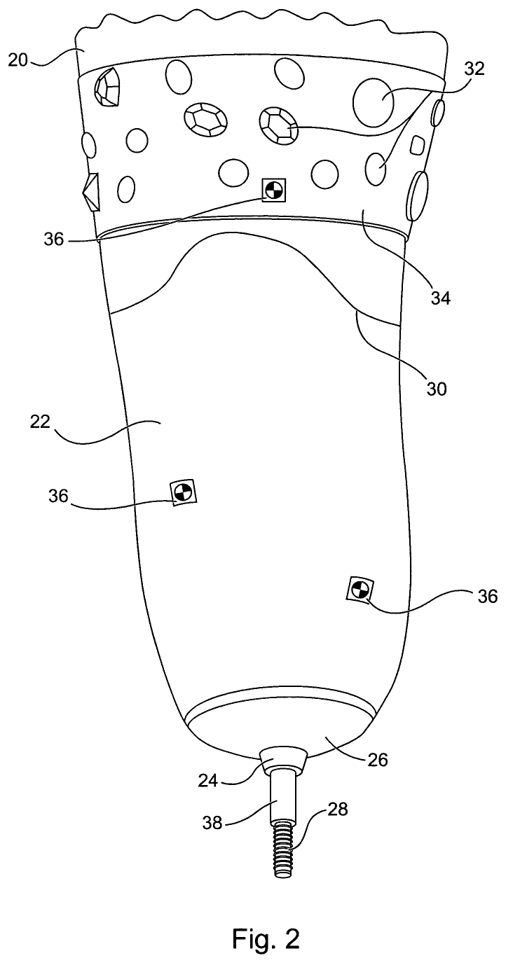

In preparation of the step of scanning the portion of the residual limb on which a prosthetic device is to be attached, a liner is preferably placed on the terminal end of the residual limb of the intended wearer. A residual limb prepared for scanning is shown in FIG. 2 and is generally represent by the numeral 20. The liner 22 is preferably the actual liner intended to be used by the wearer when wearing the prosthetic limb and includes an attachment connector 24 at its terminal end 26. The attachment connector 24 preferably comprises a corrugated rod 28 that extends downward from the liner 22.

The technician performing the scan, or other skilled person, preferably creates a marking 30 on the liner 22 that indicates the desired contour and location of the terminal edge of the socket of the prosthetic limb to be created. This marking 30 is preferably created using a piece of chalk or other erasable writing tool so that it is not permanent and can be easily changed during the scanning procedure if needed. However, it should be appreciated that any type of marking, stickers for example, could be used as an alternative.

In further preparation of the scan, artifacts 32 are also preferably positioned on the residual limb 20. The artifacts 32 preferably comprise a plurality of various objects of differing shapes and preferably have well defined edges. Preferably, the artifacts 32 also include one or more standard artifacts having very precise and known dimensions. The artifacts 32 are preferably fixed to an elastic band 34 or an adjustable belt or strap that is preferable used to hold the artifacts in the desired position relative to the residual limb 20. Preferably, the band is positioned adjacent the marking 30, opposite the terminal end 26 of the residual limb. Alternatively or additionally, optical target artifacts 36 can be positioned on the band 34 or on the liner 22. Yet further, alternatively or additionally, a standard artifact 38 of any given shape can be positioned or the rod 28 of the attachment connector 24. The band 34 of artifacts 32, the optical targets artifacts 36, and the standard artifact 38 positioned on the attachment connector 24 generally serve the same purpose and their utility is discussed below.

With the preparation complete, the residual limb is then scanned. This is preferably done using a photographic three-dimensional scanner and software, a laser three-dimensional scanner, or other three-dimensional scanner commercially available for creating digital three-dimensional representations of objects quickly and without direct contact of the object being scanned. However, alternatively, other scanning methods or devices, such as coordinate measuring machines and even hand measurements, could be used to scan an object in a manner to achieve the ultimate result of producing a digital three-dimensional representation of the object. Nonetheless, the use of a photographic three-dimensional scanner is particularly advantageous in that such a scanner is capable of obtaining data of the marking on the liner, regardless or whether the marking has any appreciable thickness or depth. This being the case, a typical photographic three-dimensional scanner produces a plurality of two-dimensional images of the residual limb along varying locations around the circumference of the limb. Using commonly available software design for use in connection with such a scanner, the two-dimensional images are automatically converted into digital representations of three-dimensional surface contours of the scanned residual limb. The three-dimensional surface contours have overlap and preferably each includes a portion that has the contour of one or more of the artifacts that were positioned on the limb during the scan. Alignment of the surface contours relative to each other is facilitated by aligning the portions of the contours associated with the artifacts. Alternatively or additionally, the optical target artifacts 36 can also be aligned using their visual edges. Additionally, the accuracy of the size of the surface contours can be calibrated using the known dimensions of the standard artifacts and comparing such dimensions to the digitally represented size of such artifacts in the scans.

Regardless of the particular scanning method utilized, it should be appreciated that a digital representation of the three-dimensional surface contour of the residual limb, with the liner thereon, is obtained. Moreover, it should be appreciated that the digital representation of the three-dimensional surface contour preferably includes information obtained from the scan regarding the desired location and contour of the terminal edge of the socket.

After the scan is complete and a digital representation of the three-dimensional surface contour of the residual limb has been created, a three-dimensional digital representation of a socket is created based thereon using commercially available computer aided design software or other suitable means. Preferably computer software scripting is used to automate this procedure. The digital representation of the three-dimensional surface contour previously created is utilized to at least partially define an interior surface of the socket that defines the socket cavity. An exterior surface of the digital representation of the socket is created that is spaced from the interior surface to form a socket wall therebetween. Additionally, the socket wall and the socket cavity are digitally represented in a manner such that they terminate at a perimeter surface that bridges the exterior and interior surfaces. Preferably, the perimeter surface is positioned and contoured based on the information regarding the desired location and contour of the terminal edge of the socket that was obtained from the scan.

Other features of the socket may also added to the three-dimensional digital representation of the socket. For example, the base of the socket may be modeled in a manner such that the socket can ultimately be attached directly to a pylon or appendage portion of the prosthetic device. To this end, the base of the socket can be digitally represented to have a fitting portion that has the features necessary for attaching the appendage portion directly to the socket without the need for separate fitting components. As other examples of optional features, the socket wall can be modeled thicker or can be made less dense at some portions of the socket relative to other portions so as to alter the flexibility, the weight, and/or the stiffness-to-weight ratio at various portions of the socket when the socket is ultimately formed of physical material. As yet another example, one or more access openings can be modeled into the digital representation of the socket that extend through the socket wall and into the cavity of the socket. Such openings can be created for purposes of venting the socket during use or for accessing a portion of the wearer's residual limb within the cavity when the prosthetic limb is worn. Furthermore, passageways running transverse to the wall thickness of the socket wall can be modeled into the socket wall to accommodate electrical wiring for heating elements or electrically controlled portions or sensors of the prosthetic device. Alternatively, the passageways can accommodate fluid for cooling the socket during use.

Once a desired socket has been fully modeled digitally in three-dimensional virtual space, the digital representation of the socket is used to drive a digitally controlled layered manufacturing device that is capable of making three-dimensional physical objects directly from such digital representations. Any type of digitally controlled layered manufacturing, such as stereo-lithography, selective laser sintering, fused deposition modeling, could be used to produce the socket directly from its three-dimensional digital representation. However, the socket is preferably formed of plastic using fused deposition modeling.

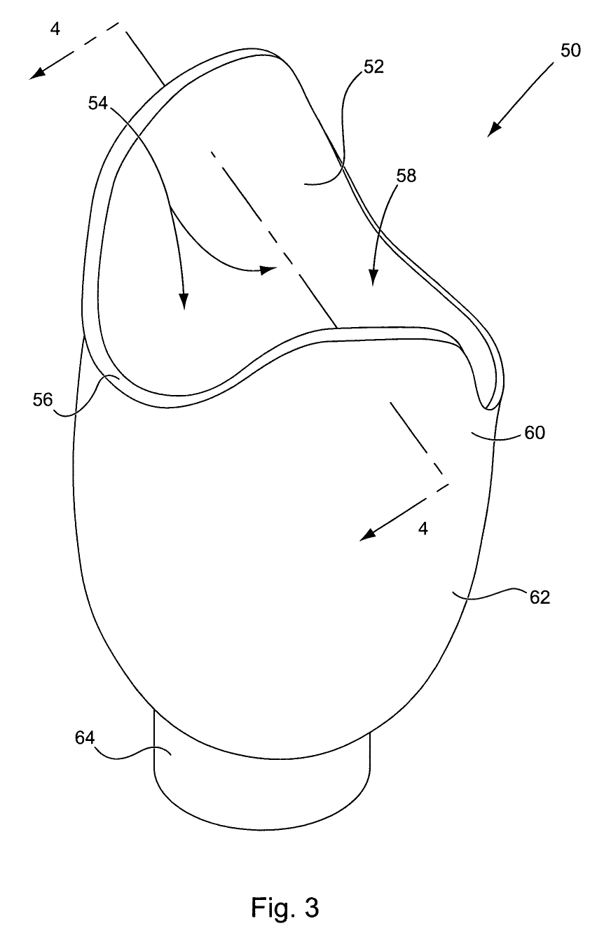

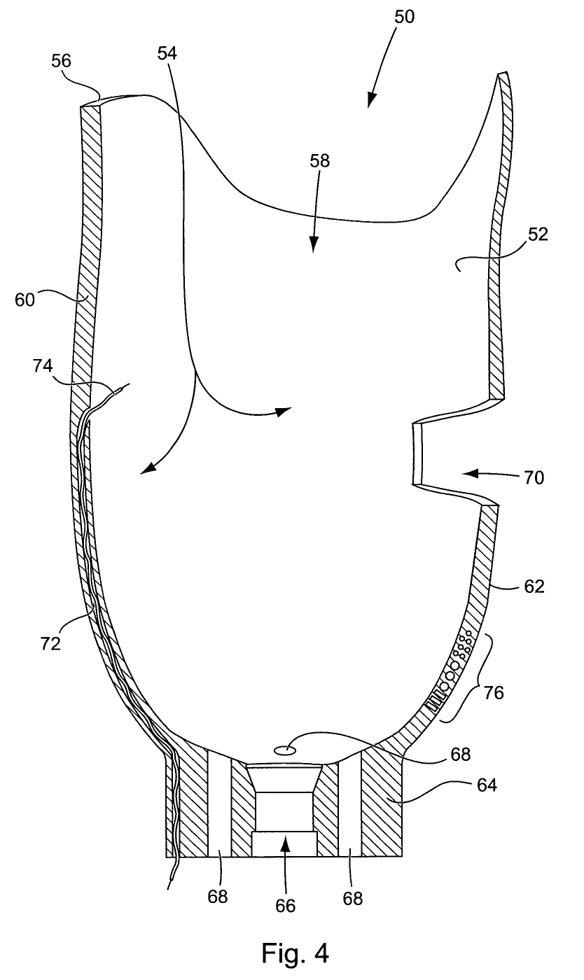

A physical socket formed by the preferred method of practicing the invention, as is shown in FIGS. 3 and 4 represented by the numeral 50, has the identical configuration of its three-dimensional digital representation and requires no appreciable fabrication steps after it has been formed by the digitally controlled layered manufacturing technique. For the purpose of further describing the examples of optional features that can be formed into the socket automatically, the socket 50 shown in FIGS. 3 and 4 includes all of the mentioned optional features. As explained, the interior surface 52 of the socket 50 has a contour matching that of a particular residual limb with a liner thereon. The interior surface 52 of the socket 50 defines the cavity 54 of the socket and terminates at a non-planar perimeter surface 56 that forms the top of the socket and that forms the primary opening 58 into the cavity. The perimeter surface 56 is contoured in a manner such that the cavity 54 of the socket 50 has a sufficient bearing surface area without impinging the movement of the residual limb by the wearer of the prosthetic limb.

The wall 60 of the socket 50 between an exterior surface 62 of the socket and the interior surface 52 varies in thickness and thereby allows for the wall stiffness to vary from one portion of the socket to another.

A fitting 64 is formed at the base of the socket 50 as an integral and homogeneous portion of the socket. A fitting opening 66 extends through the fitting 64 and is configured and adapted to allow the attachment connector of the liner to extend therethrough from within the cavity 54 of the socket 50. With the attachment connector of the liner extending through the fitting opening 66, a ratcheting connector plate or other suitable attachment device such as a treaded nut (not shown)can be used to secure the socket 50 to the liner. The fitting 64 of the socket 50 also includes a plurality of mounting holes 68 that allow an appendage portion 68 of the prosthetic limb to be connected directly to the socket 50 for securing the appendage portion to the socket without any fitting separate from that of the socket itself.

The socket 50 also includes a secondary opening 70 through the socket wall 60 that is configured to allow a portion of the residual limb within the cavity 54 of the socket to be accessible when the prosthetic limb is attached to the wearer. The same secondary opening 70 or additional secondary openings can also provide ventilation for the portion of the residual limb within the cavity 54.

The socket 50 further includes a passageway 72 within the socket wall 60 that extends transversely to the thickness of the wall and that follows the contour of the interior surface 52 of the socket. The passageway 72 allows an electrical wire 74 to be routed therethrough for use in heating the socket 50, to control various electrically controlled aspects of the prosthetic limb, or obtain sensor data from sensors mounted on the wearer's residual limb. Alternatively, the passageway 72 can be connected to a cooling fluid source and used for circulating cooling fluid to chill the socket 50, as desired for the wearer's comfort.

Yet further, the wall 60 of the socket 50 has a region 76 of reduced density relative to the remainder of the wall. This reduced density region can have many configurations such as an internal honeycomb pattern, transverse passageways, spherical voids, or virtual any shape of voids within the wall thickness. It should be appreciated that these voids provide the region 76 of the wall 60 with an increased stiffness-to-weight ratio over other portions of the wall having equal-thickness.

In view of the above, it should be appreciated that the preferred method of practicing the invention eliminates many of the steps associated with prior art methods of fabricating prosthetic limb sockets. Moreover, it should be appreciated that alterations in the configuration of a socket can easily be made by simply modifying the digital three-dimensional representation of the socket and fabricating a new socket based thereon. Thus, as compared to prior art methods, this method eliminates much of the cost, skill, and time previously needed to produce prosthetic limbs.

In addition to the advantages that the preferred method of practicing the invention has over prior art methods of manufacturing prosthetic limb sockets, the inventors of the present invention have also determined that, by forming the inner surface of the socket to conform closely to the contour of the residual limb with the liner thereon, the load bearing characteristics and wearer's comfort are also improved. This is because when a liner is placed on a residual limb, the elasticity of the liner acts to radially compress the tissue of the residual limb and, as a result, the tissue of the residual limb reshapes itself in manner such that it is more evenly compressed. In its reshaped form, the tissue of the residual limb is in a configuration that allows it to more evenly distribute radial compression forces. Thus, by scanning the residual limb with the liner thereon, the cavity of the socket is formed with the contour of the residual limb in its reshaped form and compressive radial loads are efficiently transferred from the socket to the residual limb during use of the prosthetic limb. Due to this improved socket cavity configuration, the inventors have determined that, in most situations, it is unnecessary to rectify the interior surface of the socket cavity so as to alter load distributions. Thus, the need for the highly specialized expertise normally required to configure the protuberances associated with prior art sockets is significantly diminished or altogether eliminated.

The inventors have also appreciated that, in some situations, the pylon or appendage portion of a prosthetic limb can also be formed as a contiguous and homogeneous part together with the socket of the prosthetic limb using the digitally controlled layered manufacturing technique. Likewise cosmetic prosthetic limbs could be manufactured easily by adding a scanning procedure for an opposite non-truncated limb, mirroring the digital representation of the non-truncated limb, and forming it together with the socket of the prosthetic limb. These techniques can greatly reduce the cost and labor associated with producing prosthetic limbs.

Although the invention has been described in sufficient detail to allow others to practice the present invention, it should be understood that all matter contained in the above description or shown in the accompanying drawings is intended to be interpreted as illustrative and not in a limiting sense and that various modifications and variations of the device and methods may be employed without departing from the scope of the invention defined by the following claims. For example, it should be appreciated that not all steps of the preferred method of practicing the invention are necessarily required by each claim. As another example, a socket having a non-rectified cavity contour could be formed by means other than using a digitally controlled layered manufacturing technique. Thus, with variations and modifications, other methods of practicing the invention should be appreciated.

Furthermore, it should be understood that when introducing elements of the present invention in the claims or in the above description of the preferred embodiment of the invention, the terms “comprising,” “including,” and “having” are intended to be open-ended and mean that there may be additional elements other than the listed elements. Similarly, to the extent the term “portion” is used in the claims or is added by amendment, such term should be construed as meaning some or all of the item or element that it qualifies.

What is claimed is:

1. A method of forming a prosthetic limb for attachment to a residual limb of a living animal, the method comprising:
generating a digital representation of a three-dimensional surface contour that is dependent on a physical three-dimensional surface contour of at least a portion of the residual limb;
generating a digital representation of a socket of the prosthetic limb, the socket having cavity defined by an interior surface, the interior surface being defined at least partially by the digital representation of the three-dimensional contour; and
forming the socket out of physical material using a digitally controlled layered manufacturing technique driven by the digital representation of the socket.

2. A method in accordance with claim 1 wherein the step of generating the digital representation of the socket occurs in a manner such that the socket has an exterior surface and such that the cavity of the socket terminates at a non-planar perimeter surface that bridges the exterior and interior surfaces, and wherein the step of forming the socket out of physical material occurs in a manner such that the digitally controlled layered manufacturing technique automatically forms the perimeter surface, thereby eliminating the need to form the perimeter surface by trimming the physical material.

3. A method of forming a prosthetic limb and attaching the prosthetic limb to a residual limb of a living animal comprising:
forming the prosthetic limb in accordance with claim 2, the step of forming the socket out of physical material occurring in a manner such that the digitally controlled layered manufacturing technique automatically forms an access opening in the socket that extends into the cavity from the exterior surface;
attaching the prosthetic limb to the residual limb by positioning the residual limb with the liner positioned thereon at least partially into the cavity of the socket; and
accessing a portion of the residual limb within the cavity of the socket through the access opening after the prosthetic limb has been attached to the residual limb.

4. A method in accordance with claim 1 wherein the step of generating the digital representation of the three-dimensional surface contour comprises electronically scanning the portion of the residual limb.

5. A method in accordance with claim 4 wherein the step of generating the digital representation of the three-dimensional surface contour comprises positioning at least one artifact adjacent the residual limb and wherein the scanning occurs in a manner creating a plurality of digital representations of surface contours dependent on the physical three-dimensional surface contour of the portion of the residual limb and on the artifact, the step further comprising aligning the plurality of digital representations of surface contours relative to each other by aligning portions of the plurality of digital representations of surface contours that are dependent on the artifact, the digital representation of the three-dimensional surface contour being dependent on the aligned plurality of digital representations of surface contours.

6. A method of forming a prosthetic limb and attaching the prosthetic limb to a residual limb of a living animal comprising:
forming the prosthetic limb in accordance with claim 4, the step of generating the digital representation of the three-dimensional surface contour comprising electronically scanning the portion of the residual limb with a liner positioned on the residual limb in a manner such that the interior surface is also defined at least partially by the liner; and
attaching the prosthetic limb to the residual limb by positioning the residual limb with the liner positioned thereon at least partially into the cavity of the socket.

7. A method in accordance with claim 1 wherein the step of forming the socket out of physical material using a digitally controlled layered manufacturing technique comprises using the digitally controlled layered manufacturing technique to form a fitting as an integral and homogeneous part of the socket and wherein the method further comprises:
providing a prosthetic appendage portion of the prosthetic limb that is releasably attachable directly to the fitting of the socket; and
attaching the prosthetic appendage portion directly to the fitting of the socket.

8. A method in accordance with claim 1 wherein the step of generating the digital representation of the socket of the prosthetic limb further comprises generating the digital representation of the socket in a manner such that the socket has an exterior surface that is spaced from the interior surface and that defines a socket wall that has a thickness that extends between the exterior and interior surfaces, the thickness of the socket wall varying in dimension at different portions of the socket wall, and wherein the step of forming the socket out of physical material using the digitally controlled layered manufacturing technique comprises forming the socket wall out of the physical material using the digitally controlled layered manufacturing technique.

9. A method in accordance with claim 1 wherein the step of generating the digital representation of the socket of the prosthetic limb further comprises generating the digital representation of the socket in a manner such that the socket has an exterior surface that is spaced from the interior surface and that defines a socket wall that has a thickness that extends between the exterior and interior surfaces and in a manner such that a passageway having a non-linear trajectory is formed between the interior and exterior surfaces and extends transversely to the thickness of the socket wall, and wherein the step of forming the socket out of physical material using the digitally controlled layered manufacturing technique comprises forming the socket wall and the passageway of the socket using the digitally controlled layered manufacturing technique, the method further comprising routing an electrically conductive wire within the passageway of the socket.

10. A method of forming a socket of a prosthetic limb and attaching the socket to a residual limb of a living animal comprising:
positioning a liner on at least a portion of the residual limb;
marking the liner in manner indicating a preferred contour and location of a non-planer terminal edge of the socket, the marking occurring when the liner is positioned on the residual limb;
electronically scanning at least a portion of the liner when the liner positioned on the residual limb to generate a digital representation of a three-dimensional surface contour that is dependent on a physical three-dimensional surface contour of the liner when the liner is positioned on the residual limb, the scanning occurring in a manner such that the contour and location of the non-planer terminal edge of the socket that has been marked on the liner is identifiable in the digital representation of the three-dimensional surface contour;
generating a digital representation of the socket, the digital representation of the socket having a cavity defined by an interior surface, the interior surface being defined at least partially by the digital representation of the three-dimensional contour, the digital representation of the socket also having an exterior surface and a non-planar perimeter surface, the perimeter surface terminating the cavity and bridging the exterior and interior surfaces, the perimeter surface being dependent upon the contour and location of the non-planer terminal edge of the socket that is identifiable in the digital representation of the three-dimensional surface contour;
using a digitally controlled layered manufacturing technique driven by the digital representation of the socket to form the interior, exterior, and perimeter surfaces of the socket out of physical material; and
attaching the socket of the prosthetic limb to the residual limb by positioning the residual limb with the liner positioned thereon at least partially into the cavity of the socket.

11. A method in accordance with claim 10 further comprising positioning at least one artifact adjacent the residual limb, and wherein the step of electronically scanning the portion of the liner occurs in a manner creating a plurality of digital representations of surface contours that are dependent on the physical three-dimensional surface contour of the liner and on the artifact and comprises aligning the plurality of digital representations of surface contours relative to each other by aligning portions of the plurality of digital representations of surface contours that are dependent on the artifact, the digital representation of the three-dimensional surface contour being dependent on the aligned plurality of digital representations of surface contours.

12. A method in accordance with claim 10 wherein the step of using the digitally controlled layered manufacturing technique driven by the digital representation of the socket to form the interior, exterior, and perimeter surfaces of the socket out of physical material comprises using the digitally controlled layered manufacturing technique to form a fitting as an integral and homogeneous part of the socket and wherein the method further comprises:
providing a prosthetic appendage portion of the prosthetic limb that is releasably attachable directly to the fitting of the socket; and
attaching the prosthetic appendage portion directly to the fitting of the socket.

13. A method in accordance with claim 10 wherein the step of generating the digital representation of the socket of the prosthetic limb further comprises generating the digital representation of the socket in a manner such that the exterior surface is spaced from the interior surface and defines a socket wall that has a thickness that extends between the exterior and interior surfaces, the thickness of the socket wall varying in dimension at different portions of the socket wall.

14. A method in accordance with claim 10 wherein the step of generating the digital representation of the socket further comprises generating the digital representation of the socket in a manner such that the exterior surface is spaced from the interior surface and defines a socket wall that has a thickness that extends between the exterior and interior surfaces and in a manner such that a passageway having a non-linear trajectory is formed between the interior and exterior surfaces and extends transversely to the thickness of the socket wall, and wherein the step of forming the socket out of physical material using the digitally controlled layered manufacturing technique comprises using the digitally controlled layered manufacturing technique to form the passageway of the socket, the method further comprising routing an electrically conductive wire within the passageway of the socket.

15. A method of forming a socket of a prosthetic limb and attaching the socket to a residual limb of a living animal comprising:
positioning a liner on at least a portion of the residual limb, the liner having an exterior surface contour when the liner is positioned on the portion of the residual limb;
forming a socket having an exterior surface and a cavity that is defined by an interior surface, the forming occurring in a manner such that the interior surface of the socket has a contour that is dependent upon the exterior surface contour of the liner and occurring without a process of intentionally rectifying the contour of the interior surface for the purpose of altering the bearing characteristics between the socket and the liner; and
attaching the socket of the prosthetic limb to the residual limb by positioning the residual limb with the liner positioned thereon at least partially into the cavity of the socket.

16. A method in accordance with claim 15 wherein the step of forming the socket comprises forming a fitting as an integral and homogeneous part of the socket and wherein the method further comprises:
providing a prosthetic appendage portion of the prosthetic limb that is releasably attachable directly to the fitting of the socket; and
attaching the prosthetic appendage portion directly to the fitting of the socket.

17. A method in accordance with claim 15 wherein the step of forming the socket comprises electronically scanning the exterior surface contour of the liner to generate a digital representation of the socket that is dependent upon at least a portion of the exterior surface contour of the liner, and comprises using a digitally controlled layered manufacturing technique driven by the digital representation of the socket to form the socket out of physical material.

18. A method in accordance with claim 17 wherein the step of forming the socket comprises positioning at least one artifact adjacent the residual limb prior to electronically scanning the exterior surface contour of the liner, the electronic scanning of the portion of the liner occurring in a manner creating a plurality of digital representations of surface contours that are dependent on the exterior surface contour of the liner and on the artifact, the step further comprising aligning the plurality of digital representations of surface contours relative to each other by aligning portions of the plurality of digital representations of surface contours that are dependent on the artifact, the digital representation of the socket being dependent on the aligned plurality of digital representations of surface contours.

19. A method in accordance with claim 17 wherein the step of forming the socket further comprises using the digitally controlled layered manufacturing technique to form the socket in a manner such that the exterior surface is spaced from the interior surface and defines a socket wall that has a thickness that extends between the exterior and interior surfaces, the thickness of the socket wall varying in dimension at different portions of the socket wall.

20. A method in accordance with claim 17 wherein the step of forming the socket further comprises using the digitally controlled layered manufacturing technique to form the socket in a manner such that the exterior surface is spaced from the interior surface and defines a socket wall that has a thickness that extends between the exterior and interior surfaces and in a manner such that a passageway having a non-linear trajectory is formed between the interior and exterior surfaces and extends transversely to the thickness of the socket wall, the method further comprising routing an electrically conductive wire within the passageway of the socket.

21. A prosthetic limb for attachment to a residual limb of a living animal comprising:
a socket formed by a digitally controlled layered manufacturing technique, the socket having a cavity defined by an interior surface, the interior surface being dependent on a physical three-dimensional surface contour of at least a portion of the residual limb.

22. A prosthetic limb in accordance with claim 21 wherein the socket further comprises a fitting formed as an integral and homogeneous part of the socket via the digitally controlled layered manufacturing technique.

23. A prosthetic limb in accordance with claim 21 wherein the socket has an exterior surface that is spaced from the interior surface and that defines a socket wall that has a thickness that extends between the exterior and interior surfaces, the thickness of the socket wall varying in dimension at different portions of the socket wall.

24. A prosthetic limb in accordance with claim 21 wherein the socket has an exterior surface and a non-planar perimeter surface, the perimeter surface terminating the cavity and bridging the exterior and interior surfaces, the perimeter surface being non-planar and being formed by the digitally controlled layered manufacturing technique.

25. A prosthetic limb in accordance with claim 21 wherein the socket has an exterior surface that is spaced from the interior surface and that defines a socket wall that has a thickness that extends between the exterior and interior surfaces, the socket further comprising a passageway formed between the interior and exterior surfaces, the passageway having a non-linear trajectory that extends transversely to the thickness of the socket wall, the passageway being formed by the digitally controlled layered manufacturing technique.
